(19) United States
(12) Patent Application Publication
(10) Pub. No.: US 2024/0066504 A1
(43) Pub. Date: Feb. 29, 2024

(54) METHOD FOR PRODUCING FUNCTIONAL MATERIAL MOLDED ARTICLE, FUNCTIONAL MATERIAL MOLDED ARTICLE, AND REACTOR

(71) Applicant: JAPAN AEROSPACE EXPLORATION AGENCY, Tokyo (JP)

(72) Inventors: Asuka SHIMA, Tokyo (JP); Yoshitsugu SONE, Tokyo (JP); Motohiko SATO, Kanagawa (JP); Takayuki ABE, Toyama (JP); Mitsuhiro INOUE, Toyama (JP)

(73) Assignee: JAPAN AEROSPACE EXPLORATION AGENCY, Tokyo (JP)

(21) Appl. No.: 18/261,187

(22) PCT Filed: Feb. 1, 2022
(86) PCT No.: PCT/JP2022/003788

§ 371 Date: Jul. 12, 2023

(30) Foreign Application Priority Data
Feb. 1, 2021 (JP) 2021-014563

(51) Int. Cl. B01J 23/46; B01J 12/00; B01J 19/24; B01J 21/06; B01J 35/00; B01J 35/10; B01J 37/02; C07C 1/12

(52) U.S. Cl. CPC B01J 23/462; B01J 12/007; B01J 19/24; B01J 21/063; B01J 35/0013; B01J 35/1009; B01J 35/1014; B01J 37/0203; B01J 37/0236; C07C 1/12

(57) ABSTRACT

Provided is a technique for molding a functional material without deteriorating the function of the functional material. A method for producing a functional material molded article of the present disclosure includes: dispersing a functional material in a water-alcohol mixed solution to obtain a liquid dispersion; impregnating a porous molding base material with the liquid dispersion to obtain an impregnated product; and drying the impregnated product.

### TECHNICAL FIELD

The present disclosure relates to a method for producing a functional material molded article, a functional material molded article, and a reactor.

### BACKGROUND ART

While the application of renewable energy is expected to expand, technologies using renewable energy for generating carriers capable of storing and transporting energy are being actively studied. For example, it has been studied to generate hydrogen by electrolyzing water using renewable energy and to use this hydrogen as a thermal energy source or fuel for a fuel cell. In addition, the use of hydrogen by converting it to methane or ammonia has also been studied. In particular, methane is expected to be used as an energy carrier since it is a main component of natural gas and the existing infrastructure can be used, which is an advantage.

The Sabatier reaction is known as a technique for converting hydrogen into methane. This Sabatier reaction is a technique to produce methane and water from a catalytic reaction between hydrogen and carbon dioxide. The Sabatier reaction is a reaction in which the reduction rate of carbon dioxide by hydrogen reaches nearly 100% at a temperature of 350° C., and carbon dioxide can be reduced by hydrogen with high efficiency. Further, the Sabatier reaction is an autonomic reaction accompanied by heat generation, and the reaction can be continued without supplying thermal energy or the like from the outside.

As a catalyst for the Sabatier reaction, Patent Literature 1 discloses a hydrogen reduction catalyst for carbon dioxide in which catalytic metal nanoparticles and metal oxide particles are dispersed and supported on a powdery carrier. For such a powder catalyst, it is desired to establish a secondary molding method.

Various methods for molding functional material powder, such as a catalyst, to form a larger structure are known, such as a method for compressing a functional material, a method for granulating a functional material by mixing it with an adhesive such as a binder, and a method for bonding a functional material to a structure to which an adhesive or the like is applied in advance.

Patent Literature 2 discloses a method for producing a carrier-supported solid catalyst for producing aldehydes in which a carrier made of a through hole-type porous material supports a catalyst component. The method disclosed in Patent Literature 2 mixes a catalyst with water to impregnate a porous body with the mixture and performs drying and sintering, thereby obtaining a molded article (see Example 1 of Patent Literature 2).

Patent Literature 3 discloses a process for supporting a catalyst power containing a complex metal oxide having molybdenum as an essential ingredient on an inert support by a tumbling granulation method. The process disclosed in Patent Literature 3 uses a binder (see Example 1 of Patent Literature 3).

### CITATION LIST

### Patent Literature

Patent Literature 1: JP 2019-048249 A
Patent Literature 2: JP 2017-047377 A
Patent Literature 3: WO 2013/161703

### SUMMARY OF INVENTION

### Technical Problem

Unfortunately, it is difficult to apply the molding method through a sintering step as disclosed in Patent Literature 2 to the Sabatier catalyst disclosed in Patent Literature 1 since overheating by the sintering causes deterioration of the Sabatier catalyst performance due to its functional property. Since molding using a binder as in Patent Literature 3 causes covering the surface of the functional material such as a catalyst, the function of the functional material may be reduced.

The present disclosure provides a technique for molding a functional material without deteriorating the function of the functional material.

### Solution to Problem

To solve the above issues, a method for producing a functional material molded article of the present disclosure includes: dispersing a functional material in a water-alcohol mixed solution to obtain a liquid dispersion: impregnating a porous molding base material with the liquid dispersion to obtain an impregnated product; and drying the impregnated product.

Further features relating to the present disclosure will become apparent from the descriptions in the present specification and the attached drawings. Aspects of the present disclosure may be achieved or implemented by elements, various combinations of such elements, the following detailed descriptions, and the appended claims. The descriptions in the present specification are for exemplary purposes only, and do not in any way represent a limitation of the scope of the claims or application examples of the present disclosure.

### Advantageous Effects of Invention

According to the technique of the present disclosure, it is possible to mold a functional material without deteriorating the function of the functional material. Other issues, configurations, and advantageous effects will become apparent from the following description of an embodiment.

### BRIEF DESCRIPTION OF DRAWINGS

FIG. 1 is a flowchart of a method for producing a functional material molded article according to an embodiment of the present disclosure.

FIG. 2 is a schematic diagram of a porous molding base material before impregnation with a functional material liquid dispersion and a functional material molded article.

FIG. 3 is a cross-sectional schematic diagram illustrating a configuration of a part of a reactor including a functional material molded article.

FIG. 4 is an enlarged photograph of an alumina plate.

FIG. 5 is a photograph of an alumina plate and a powder catalyst molded article.

FIG. 6 is an enlarged photograph of a powder catalyst molded article according to Example 1.

FIG. 7 is a photograph of a reactor in which 15 powder catalyst molded articles are filled in a reaction cell.

FIG. 8 is a graph illustrating catalyst performances of the powder catalyst molded articles according to Example 1 and Comparative Example 1.

FIG. 9 is a photograph of a reactor of a flat plate structure according to Example 3.

FIG. 10 is a graph illustrating catalyst performances of the powder catalyst molded articles according to Example 1 and Example 3.

### DESCRIPTION OF EMBODIMENTS

[Method for Producing Functional Material Molded Article]

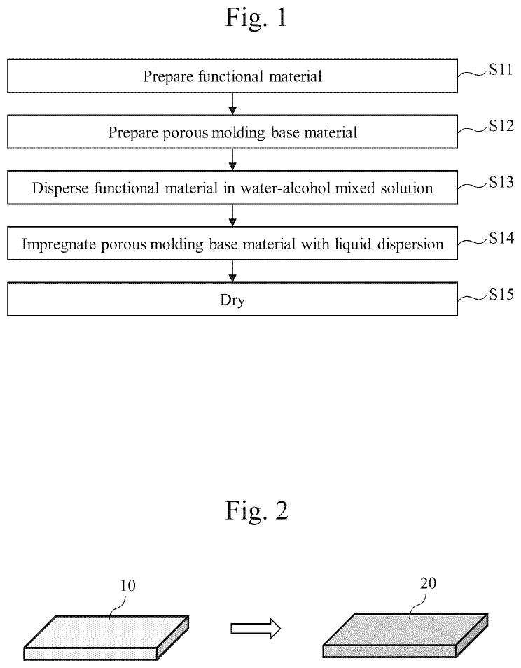

FIG. 1 is a flowchart of a method for producing a functional material molded article according to an embodiment of the present disclosure.

<Step S11>

A producer prepares a functional material intended to be molded. The functional material is in the form of powder or particles. Examples of the functional material include a catalyst and an adsorbent. Examples of a material that forms the catalyst include a metal and a metal oxide exhibiting a catalytic activity. Examples of a material that forms the adsorbent include silica gel, carbon, zeolite, resin, or mineral.

The catalyst may be a particulate catalyst or a powder catalyst in which catalyst particles are supported on carrier particles. Other than the catalyst particles, particles of another material for maintaining the function of the catalyst may be supported on the carrier particles. As a hydrogen reduction catalyst (Sabatier catalyst) for carbon dioxide, Patent Literature 1 discloses a powder catalyst in which catalytic metal nanoparticles and metal oxide particles for suppressing grain growth of the catalytic metal nanoparticles are dispersed and supported on a carrier.

The catalytic metal nanoparticles of the hydrogen reduction powder catalyst for carbon dioxide are, for example, nanoparticles containing at least one type of metal selected from the group consisting of Fe, Co, Ni, Cu, Ru, Pd, Ag, Ir, and Pt. The catalytic metal nanoparticles may contain a metal oxide as long as the catalyst function is not impaired.

The metal oxide particles of the hydrogen reduction powder catalyst for carbon dioxide are formed of a metal oxide that is not easily changed by heating in the presence of hydrogen and has high reduction resistance. The metal oxide includes, for example, at least one type of metal oxide selected from the group consisting of titanium dioxide and zirconium dioxide. The metal oxide may contain a catalytic metal as long as the reduction resistance is not impaired. Each of these metal oxides may be used alone, or two of these may be used in combination.

As a carrier of the hydrogen reduction powder catalyst for carbon dioxide, for example, silicon dioxide, magnesium oxide, titanium dioxide, zirconium dioxide, diniobium pentoxide, zeolite or calcium phosphate may be used. One type of these carriers may be used alone, or two or more types thereof may be used in combination. The carrier may have a spherical shape, a polyhedron shape, an irregular shape, a flake shape, or a scale shape. The average particle diameter of the carrier may be, for example, 0.01 μm to 30 μm or 0.02 μm to 2.0 μm, which is not particularly limited thereto.

A powder catalyst may be produced by performing sputtering while rolling a carrier using a target containing the above metal and metal oxide. This allows nanoparticles containing the metal and nanoparticles containing the metal oxide to be dispersed and supported on the surface of the carrier. As an apparatus for performing sputtering while rolling the carrier, for example, a polygonal barrel sputtering apparatus may be used.

<Step S12>

The producer prepares a porous molding base material. The shape of the porous molding base material is not particularly limited, and may be, for example, a plate shape, a disc shape, a rectangular parallelepiped shape, a cubic shape, a spherical shape, a hemispherical shape, a pyramidal shape, a conical shape, a cylindrical shape, or a combination thereof. The shape of the porous molding base material may be selected according to the shape of the functional material after being molded. In particular, when the Sabatier catalyst is molded, thermal stability may be improved by forming the porous molding base material into a plate shape and then thinning a reaction region.

Examples of a material of the porous molding base material include porous ceramic and porous metal. Immobilization of the functional material can be facilitated by using a material having affinity with the functional material for the material of the porous molding base material.

Examples of ceramic that serves as a raw material for porous ceramic include oxides such as alumina, zirconia, and barium titanate; hydroxides such as hydroxyapatite; carbides such as silicon carbide; nitrides such as silicon nitride; halides such as fluorite; phosphates; and carbonates, which are not limited thereto.

For a metal that serves as a raw material for porous metal, a single metal and an alloy may be used, such as titanium, copper, and SUS, for example.

The size of a pore of the porous molding base material may be equal to or larger than the particle diameter of the functional material. This allows the functional material to enter the pores of the porous molding base material and to be retained therein.

By adjusting the porosity of the porous molding base material, the amount of the functional material retained by the porous molding base material per unit volume can be adjusted, thereby reactivity can be controlled. The porous molding base material may have a porosity of 10% to 90%, 20% to 80%, or 30% to 70%, for example, though not limited thereto.

The porous molding base material may have a specific surface area of 0.5 to 10 m²/g or 1.0 to 5.0 m²/g, for example.

<Step S13>

The producer adds the functional material to a water-alcohol mixed solution and disperses the functional material in the water-alcohol mixed solution to obtain a liquid dispersion (including one in the form of slurry). The mixing ratio (volume ratio) between water and alcohol may be 5:95 to 95:5, 10:90 to 90:10, 20:80 to 80:20, or 30:70 to 70:30, for example.

Examples of alcohol include methanol, ethanol, n-propanol, isopropanol, n-butanol, ethylene glycol, propylene glycol, and butanediol, which are not limited thereto. In particular, use of alcohol that makes the water-alcohol mixed solution an azeotropic mixture allows the functional material to be stably retained by the porous molding base material since the mixed solution will evaporate without change in the composition of the water-alcohol mixed solution in the drying step that will be described later.

The mixing ratio (weight ratio) between the water-alcohol mixed solution and the functional material may be 20:80 to 80:20, 30:70 to 70:30, or 40:60 to 60:40, for example. In particular, the mixing ratio of 70:30 to 80:20 provides excellent operability during impregnation, depending on the type of functional material and the concentration of water-alcohol mixed solution.

To the water-alcohol mixed solution, any additive may be added as long as the function of the functional material is not impaired.

<Step S14>

The producer impregnates the porous molding base material with the liquid dispersion to obtain an impregnated product. The liquid dispersion enters the pores of the porous molding base material by capillarity, and thus can reach the inner part of the porous molding base material. The impregnation method is not particularly limited. Examples of the impregnation method include a method for dropping a liquid dispersion onto a porous molding base material and a method for immersing a porous molding base material in a liquid dispersion filled into a container.

The impregnation amount of the liquid dispersion may be 50 to 300 mg/cm³, 100 to 250 mg/cm³, or 150 to 200 mg/cm³, for example.

It should be noted that the entire porous molding base material may be uniformly impregnated with the liquid dispersion or the porous molding base material may be impregnated with the liquid dispersion in an unbalanced manner. That is, there may be a gradient in the amount of functional material retained by the porous molding base material. For example, the amount of functional material may be changed from one end to the other end of the porous molding base material or the amount of functional material may be changed in a concentric manner.

<Step S15>

The producer dries the porous molding base material impregnated with the liquid dispersion to remove the water-alcohol mixed solution, thereby producing a functional material molded article. Drying may be performed naturally or with a dryer. The drying temperature may be any temperature as long as the function of the functional material is not impaired, for example, temperatures between room temperature and 300° C. Since the Sabatier catalyst used in Examples, which will be described later, exhibits a high catalytic activity at 220° C., for example, the drying temperature may be equal to or lower than 220° C. to avoid deterioration of the performance due to heating over 220° C. When the function of the functional material will not be reduced by heating at high temperatures, drying may be performed at a temperature equal to or higher than an operating temperature of the functional material.

According to the method for producing a functional material molded article of the present embodiment, the functional material may be retained in the pores of the porous molding base material. When the entire porous molding base material is impregnated with the liquid dispersion, the functional material is evenly retained within the pores, thus achieving retention of the functional material in a large amount. This is effective for improvement of the reactivity of the reaction using the functional material molded article. The amount of the functional material retained by the functional material molded article may be 50 to 300 mg/cm³, 80 to 250 mg/cm³, or 100 to 230 mg/cm³, for example, depending on the weight of the functional material and the porosity of the porous molding base material.

Steps S14 and S15 may be carried out multiple times. The produced functional material molded article may be sintered as long as the function of the functional material is not impaired. The sintering temperature may be set according to the type of functional material. Note that, when the functional material is the Sabatier catalyst, sintering is not performed so as to maintain the catalyst performance since the Sabatier catalyst exhibits activity at a temperature of 220° C. or lower.

FIG. 2 is a schematic diagram of a porous molding base material 10 before impregnation with a functional material liquid dispersion and a functional material molded article 20. As illustrated on the left side of FIG. 2, the porous molding base material 10 has a plate shape in one example. As illustrated on the right side of FIG. 2, the produced functional material molded article 20 is molded into a shape substantially equal to the shape of the porous molding base material 10. The functional material molded article 20 may be processed in other shapes by cutting or the like, for example.

<Conclusion>

As described above, the method for producing the functional material molded article according to the present embodiment includes dispersing a functional material in a water-alcohol mixed solution to obtain a liquid dispersion, impregnating a porous molding base material with the liquid dispersion to obtain an impregnated product, and drying the impregnated product. This allows molding the functional material without using a binder, and thus the binder will not cover the functional material. Accordingly, since the functional material can be molded while maintaining the surface area of the functional material, reduction of the function of the functional material can be avoided. In addition, by employing an azeotropic mixture for the water-alcohol mixed solution, the mixed solution will evaporate at a temperature lower than the boiling point of water, and thus a lower drying temperature can be set. Accordingly, since a heat load to the functional material can be reduced, it is possible to avoid reduction of the function caused by heat. Furthermore, since the functional material can be molded without sintering, irreversible deterioration of the functional material caused by overheating can be avoided in advance.

[Reactor]

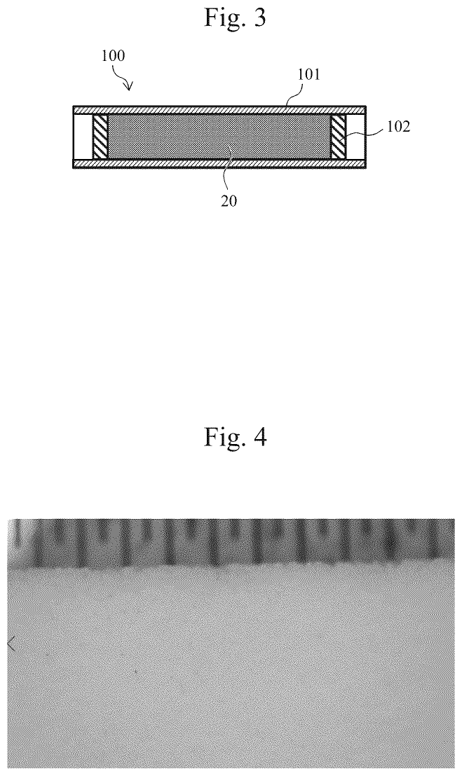

FIG. 3 is a cross-sectional schematic diagram illustrating a configuration of a part of a reactor 100 including the functional material molded article 20. As illustrated in FIG. 3, the reactor 100 includes the functional material molded article 20, a reaction cell 101, and a porous material 102.

The reaction cell 101 is open at both ends, and its inner space is filled with the functional material molded article 20. The number of functional material molded articles 20 filled in the reaction cell 101 may be one or more. One of the openings of the reaction cell 101 is coupled with a raw material supply pipe (not illustrated) for supplying a raw material (gas or liquid) for reaction. The other of the openings of the reaction cell 101 is coupled with a discharge pipe (not illustrated) for discharging a reaction product (gas or liquid). The shape of the reaction cell 101 is not particularly limited, and may be, for example, a cylindrical shape, an elliptical cylindrical shape, or a polygonal cylindrical shape. The material of the reaction cell 101 is not particularly limited as long as it is not reactive with supplied raw materials and discharged reaction products and it has a resistance to a reaction temperature. As a material of the reaction cell 101, for example, plastic, metal, ceramic, glass or the like may be used.

The porous material 102 allows gas and liquid such as carbon dioxide, hydrogen, methane and water (water vapor) to pass through without passing through the functional material molded article 20. As the porous material 102, for example, a metal fiber filter, a ceramic filter, a glass filter, a foam metal, or glass wool may be used.

The reactor 100 may include a heater for adjusting a reaction temperature. The reactor 100 may also include a thermometer for measuring the internal temperature of the reaction cell 101.

When the functional material is a catalyst, the reactor 100 may be used as a catalytic reactor. In particular, when the functional material is the Sabatier catalyst, the reactor 100 may be used as a hydrogen reduction device for carbon dioxide. In this case, the reaction cell 101 is supplied with carbon dioxide and hydrogen as raw material gases, and then methane and water are discharged as reaction products.

When the functional material is an adsorbent, the reactor 100 may be used as an adsorption reactor.

### EXAMPLES

Hereinafter, examples of the technique of the present disclosure will be described.

### Experimental Example 1: Production of Powder Catalyst Molded Article

### Example 1

<Production of Powder Catalyst>

For the functional material, a powder catalyst (Sabatier catalyst) for hydrogen reduction of carbon dioxide was prepared. The powder catalyst was produced in the following procedure.

A Ru target as a metal target, and a ZrO₂ target as a metal oxide target were placed in a target holder of the polygonal barrel sputtering apparatus. The area ratio of the sputtering surfaces of the Ru target and the ZrO₂ target placed in the target holder was 1:0.5. The target holder was tilted so that the sputtering surfaces of the Ru target and the ZrO₂ target faced downward.

As a carrier, 3.0 g of TiO₂ powder (anatase type) was introduced into an octagonal barrel of the polygonal barrel sputtering apparatus. The TiO₂ powder had an average particle diameter of 100 nm.

Subsequently, the pressure inside the octagonal barrel was reduced to 8.0× 10⁻⁴ Pa or less using a rotary pump and an oil diffusion pump. Thereafter, Ar gas was introduced into the octagonal barrel by an argon gas introduction mechanism, and the pressure inside the octagonal barrel was set to 0.8 Pa. Then, the octagonal barrel was swung by a rotation mechanism at an angle of 75° and 4.3 rpm to agitate the TiO₂ powder in the octagonal barrel. While the agitation, a high frequency of 100 W was applied to a high frequency application mechanism (RF oscillator) for 12 hours to obtain a TiO₂ granular material (Ru—ZrO₂/TiO₂) supporting Ru—ZrO₂. This TiO₂ granular material supporting Ru—ZrO₂ was the “powder catalyst.” The powder catalyst was black, with a structure in which ruthenium (Ru) and zirconium oxide (ZrO₂) were dispersed and supported on the titanium dioxide (TiO₂) carrier.

The amount of Ru supported on the powder catalyst was confirmed by X-ray fluorescence analysis, and was 23.3 wt %. The amount of ZrO₂ supported on the powder catalyst was about 3.5 wt % as an estimate value. The particle diameter of the Ru particles supported on the powder catalyst was 0.4 to 3.0 nm, and the average particle diameter was 1.3 nm (n=142).

<Formulation of Powder Catalyst Slurry>

25% of water and 75% of isopropanol (IPA) by volume ratio were mixed to obtain a water-IPA mixed solution. The water-IPA mixed solution and the powder catalyst were mixed in a mixing ratio (weight ratio) of 10:1 to disperse the powder catalyst, and a slurry was obtained.

<Preparation of Ceramic Porous Body>

A ceramic porous body was used as a porous molding base material. 15 porous alumina (Al₂O₃) plates (available from Lepton, Inc.) were prepared as the ceramic porous body. The alumina plate had a dimension of 1.9 cm (length)×1.2 cm (width)×0.2 cm (height), a porosity of 30% to 60%, and a specific surface area of 1 to 3 m²/g. FIG. 4 is an enlarged photograph of the alumina plate. As illustrated in FIG. 4, it can be confirmed that the porous alumina plate is white and has fine pores.

<Production of Molded Article>

A part of the powder catalyst slurry was dropped onto the alumina plate, and the alumina plate was impregnated with the slurry by capillarity. Then the alumina plate was dried at a temperature of 100° C. or lower (30 to 50° C.) for 30 to 60 minutes in an oven (thermostatic drying furnace). This step was repeated multiple times and powder catalyst molded articles were obtained. Note that 15 alumina plates were impregnated with the slurry and dried, and 15 powder catalyst molded articles were obtained in the same manner. The alumina plate was impregnated with the powder catalyst slurry in an amount of 150 to 200 mg/cm³. The amount of the powder catalyst retained by the obtained powder catalyst molded article was 42 to 51 mg.

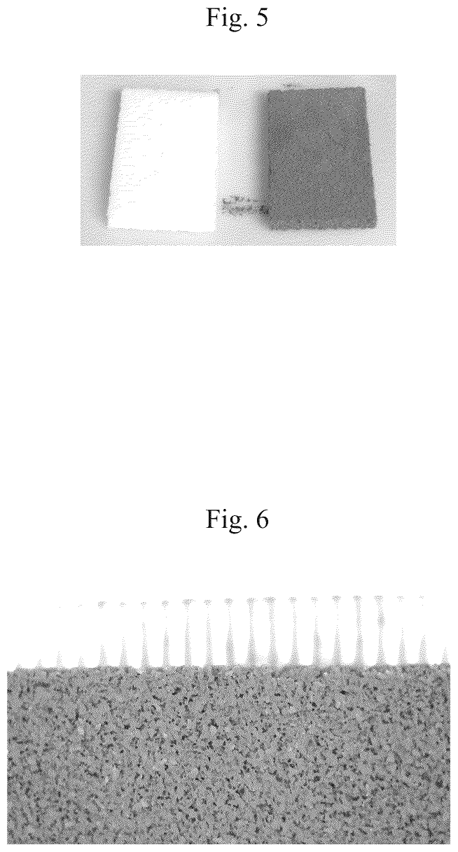

FIG. 5 is a photograph of the alumina plate and the powder catalyst molded article. As illustrated in FIG. 5, the alumina plate (left) before impregnation with the slurry was white and the powder catalyst molded article (right) obtained by being impregnated with the slurry and dried was gray. The shape of the powder catalyst molded article had no substantial change from the shape of the alumina plate, and thus it was found that the powder catalyst was moldable into the shape of the porous molding base material. FIG. 6 is an enlarged photograph of the powder catalyst molded article. As illustrated in FIG. 6, it is found that the black powder catalyst was immobilized and retained in the fine pores of the alumina plate.

### Comparative Example 1

<Production of Molded Article>

2 g of glass wool (molding base material) and 0.7 g of powder catalyst produced in the same manner as in Example 1 were mixed to disperse the powder catalyst, thereby a powder catalyst molded article according to Comparative Example 1 was obtained.

[Evaluation of Moldability]

Moldability of the powder catalyst molded articles according to Example 1 and Comparative Example 1 was evaluated as follows.

<Operability During Immobilization onto Base Material>

Example 1 . . . . It worked without problems during impregnation with the slurry.

Comparative Example 1 . . . . During dispersion in glass wool, desorption of the powder catalyst occurred and the operability was lower than in Example 1.

<Immobilized State of Powder Catalyst of Molded Article>

Example 1 . . . . The powder catalyst was immobilized onto the entire alumina plate, and desorption or dusting of the powder catalyst did not occur.

Comparative Example 1 . . . . Desorption of the powder catalyst partially occurred.

<Conclusion>

As described above, implementing the method for producing a powder catalyst molded article according to Example 1, the powder catalyst could be easily immobilized onto the alumina plate and molded. This method does not use a binder, and thus the powder catalyst will not be covered by the binder. Therefore, it can be said that the catalyst function of the powder catalyst is maintained.

[Evaluation of Catalyst Performance]

A hydrogen reduction reaction of carbon dioxide was carried out using the powder catalyst molded articles according to Example 1 and Comparative Example 1, and the catalyst performance was evaluated as follows.

<Filling Powder Catalyst Molded Articles into Reactor>

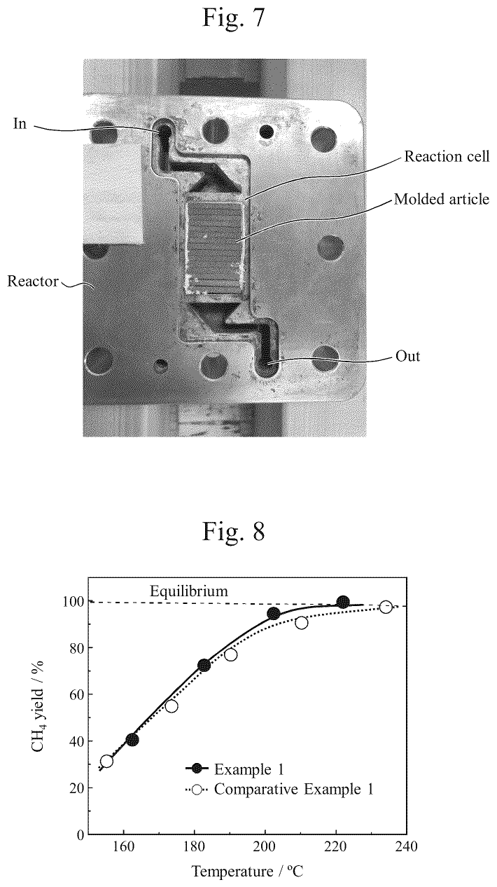

15 powder catalyst molded articles according to Example 1 were stacked and then filled, as a columnar catalyst layer of 1.9 cm×1.2 cm×2.8 cm, in the reaction cell of the reactor. The volume of the internal space of the reaction cell was 6 cm² (cross sectional area)×3.2 cm (height)=19.2 cm³. The amount of the powder catalyst retained by the columnar catalyst layer was 0.67 g. A small gap between the inside dimension of the reaction cell and the columnar catalyst layer was filled with a filler containing alumina as a main component. FIG. 7 is a photograph of the reactor in which 15 powder catalyst molded articles are filled in the reaction cell. As illustrated in FIG. 7, in the reaction cell, the powder catalyst molded articles each are placed perpendicularly to a gas traveling direction and are stacked along the gas traveling direction.

Thermocouples were placed at three locations of the filled columnar catalyst layer. A heater was disposed in the reactor, and the upper opening of the reaction cell was coupled with the gas supply pipe equipped with a mass flowmeter (MFC). The lower opening of the reaction cell was coupled with the discharge pipe.

The powder catalyst molded articles according to Comparative Example 1 were filled in the reaction cell of the reactor. The volume of the internal space of the reaction cell was 19.2 cm³, and the amount of the powder catalyst retained by glass wool was 0.7 g. Thus, the amount of the powder catalyst retained by glass wool was 0.036 g/cm³. The other conditions were the same as those of the above Example 1 to prepare the reactor.

<Hydrogen Reduction Reaction of Carbon Dioxide>

For each of Example 1 and Comparative Example 1, a gas mixture of CO₂ 10 mL/min and H₂ 40 mL/min was supplied to the reactor while heating the powder catalyst molded articles to a predetermined temperature by driving the heater. The temperatures of the powder catalyst molded articles were measured by the thermocouples. When the measurements by the respective thermocouples became substantially constant, the gas after passing through the powder catalyst molded articles was sampled.

After the sampling, the set temperature of the heater was changed, and in the same manner as the above, CO₂/H₂ gas mixture was supplied to the reaction cell, and the gas after passing through the powder catalyst molded articles was sampled. The set temperatures of the heater in Example 1 were 160° C., 180° C. 200° C., and 220° C. The set temperatures of the heater in Comparative Example 1 were 160° C., 180° C., 200° C., 220° C., and 240° C.

The sampled gas was analyzed using gas chromatography, and reaction conversion was calculated from the peaks of CH₄ (product) and CO₂ (unreacted raw material) in the analysis chart. Note that when comparison with the result of other conditions was made using graphs and the like, the obtained conversion was expressed as a function of the maximum temperature of the measured catalyst.

<Results>

FIG. 8 is a graph illustrating catalyst performances of the powder catalyst molded articles according to Example 1 and Comparative Example 1. The horizontal axis denotes the measured temperature and the vertical axis denotes the yield of CH₄. The measured temperatures in Example 1 were 163° C., 183° C. 203° C., and 222° C. The measured temperatures in Comparative Example 1 were 155° C., 174° C., 190° C., 210° C., and 234° C. As illustrated in FIG. 8, the yield of CH₄ in Example 1 was 40.4% when the reaction temperature was 163° C., and the yield increases as the reaction temperature rises, and the yield of CH₄ was 72.3% at 183° C., 94.5% at 202° C., and 99.4% at 222° C. The yield of CH₄ in Comparative Example 1 was 31.3% when the reaction temperature was 155° C., the yield increases as the reaction temperature rises, and the yield of CHₐ was 90.5% at 210° C., 97.2% at 234° C. The approximate curves in Example 1 and Comparative Example 1 illustrated in FIG. 8 are similar. Therefore, it is found that the catalyst performance of the powder catalyst molded article according to Example 1 is equal to the catalyst performance of the powder catalyst molded article according to Comparative Example 1 at each reaction temperature, and the function of the powder catalyst molded article as a catalyst is maintained by the method of Example 1.

### Example 2

In the same manner as in Example 1, a powder catalyst molded article in which a powder catalyst is retained by an alumina plate was produced. The amount of the powder catalyst retained by the powder catalyst molded article obtained in Example 2 was 70 mg. In this manner, 70 mg of powder catalyst could be retained by the alumina plate of 1.9 cm×1.2 cm×0.2 cm=0.456 cm³. That is, a retaining amount of 153.5 mg/cm³ was achieved.

### Experimental Example 2: Change in Shape of Catalyst Layer

### Example 3

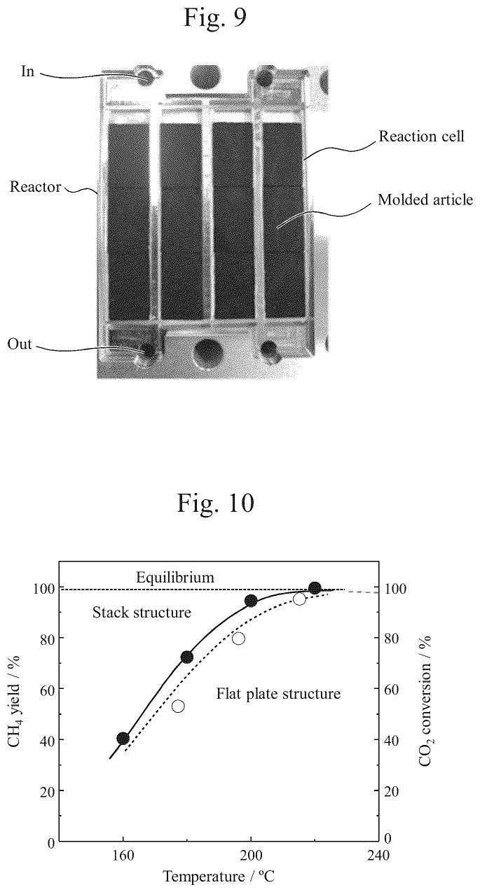

3 (length)×4 (width) (12 in total) pieces of the powder catalyst molded articles according to Example 1 were arranged on a plane to produce a reactor having a flat plate structure. FIG. 9 is a photograph of the reactor of the flat plate structure according to Example 3.

In the same manner as in Example 1, using the reactor according to Example 3, CO₂/H₂ gas mixture was supplied and the gas after passing through the catalyst layer was sampled.

FIG. 10 is a graph illustrating catalyst performances of the powder catalyst molded articles according to Example 1 and Example 3. As illustrated in FIG. 10, as compared with Example 1, the yield of CH₄ in Example 3 is slightly low, but is substantially equal to that of Example 1. Therefore, it is found that the catalyst performance is maintained both when the powder catalyst molded articles are stacked (Example 1) and when the powder catalyst molded articles are arranged on the plane (Example 3).

### Experimental Example 3: Change in Concentration of Water-Alcohol Mixed Solution

### Example 4

The conditions of Example 4 were the same as those of Example 1 except that the mixing ratio (volume ratio) of water-IPA mixed solution was water 50%:IPA 50%, and a powder catalyst molded article according to Example 4 was produced.

### Example 5

The conditions of Example 5 were the same as those of Example 4 except that the mixing ratio of water-IPA mixed solution was water 80%:IPA 20%, and a powder catalyst molded article according to Example 5 was produced.

### Example 6

The conditions of Example 6 were the same as those of Example 4 except that the mixing ratio of water-IPA mixed solution was water 20%:IPA 80%, and a powder catalyst molded article according to Example 6 was produced.

### Comparative Example 2

The conditions of Comparative Example 2 were the same as those of Example 4 except that the mixing ratio of water-IPA mixed solution was water 100%:IPA 0%, and a powder catalyst molded article according to Comparative Example 2 was produced.

### Comparative Example 3

The conditions of Comparative Example 3 were the same as those of Example 4 except that the mixing ratio of water-IPA mixed solution was water 0%:IPA 100%, and a powder catalyst molded article according to Comparative Example 3 was produced.

[Evaluation of Moldability]

Moldability of the powder catalyst molded articles according to Examples 4 to 6 and Comparative Examples 2 and 3 was evaluated as follows.

<Operability During Impregnation with Slurry onto Base Material>

Example 4 . . . . It worked without problems during impregnation with the slurry.

Example 5 . . . . It worked without problems during impregnation with the slurry.

Example 6 . . . . It worked without problems during impregnation with the slurry, but the operability was slightly lower than in Examples 4 and 5.

Comparative Example 2 . . . . The slurry (liquid water dispersion of the powder catalyst) hardly adhered to the alumina plate.

Comparative Example 3 . . . . The slurry (liquid IPA dispersion of the powder catalyst) dried so quickly that operation failed.

<Immobilized State of Powder Catalyst of Molded Article>

Example 4 . . . . The powder catalyst was immobilized onto the entire alumina plate, and desorption or dusting of the powder catalyst did not occur.

Example 5 . . . . The powder catalyst was immobilized onto the entire alumina plate, and desorption or dusting of the powder catalyst did not occur.

Example 6 . . . . The powder catalyst was immobilized onto the entire alumina plate, and desorption or dusting of the powder catalyst did not occur.

Comparative Example 2 . . . . A small amount of powder catalyst was immobilized.

Comparative Example 3 . . . . A small amount of powder catalyst was immobilized.

<Immobilized State of Powder Catalyst after Reaction>

In the same manner as in Example 1, a hydrogen reduction of carbon dioxide was carried out using the powder catalyst molded articles according to Examples 4 to 6. After that, the immobilized state of powder catalyst was confirmed.

Example 4 . . . . The powder catalyst was immobilized onto the entire alumina plate, and desorption or dusting of the powder catalyst did not occur.

Example 5 . . . . The powder catalyst was immobilized onto the entire alumina plate, and desorption or dusting of the powder catalyst did not occur.

Example 6 . . . . The powder catalyst was immobilized onto the entire alumina plate, and desorption or dusting of the powder catalyst did not occur.

### Experimental Example 4: Use of Binder and Use of Metallic Molding Base Material

### Comparative Example 4

<Production of Powder Catalyst Molded Article>

For the porous molding base material, a SUS porous body (available from NAGAMINE MANUFACTURING Co., Ltd., MF-55) was prepared. The SUS porous body was made from SUS316L, and had a cell density of 55 PPI, an average pore size of 0.20 mm, and an average pore rate of 86%.

The powder catalyst produced in Experimental Example 1 and an alumina-based adhesive as a binder were mixed, and a slurry was obtained. This slurry was applied to the porous SUS and dried for 30 minutes at 120° C. or lower, and a powder catalyst molded article according to Comparative Example 4 was obtained.

### Comparative Example 5

The conditions of Comparative Example 5 were the same as those of Comparative Example 4 except that a titanium porous body (material: Ti) was used for a porous molding base material, and a powder catalyst molded article according to Comparative Example 5 was produced.

### Comparative Example 6

The conditions of Comparative Example 6 were the same as those of Comparative Example 4 except that a copper porous body (material: Cu) was used for a porous molding base material, and a powder catalyst molded article according to Comparative Example 6 was produced.

[Evaluation of Catalyst Performance]

In the same manner as in Example 1, hydrogen reduction reaction of carbon dioxide was carried out using the powder catalyst molded articles according to Comparative Examples 4 to 6. As a result, it was found that the yield of CH₄ was low and reaction hardly occurred in all of Comparative Examples 4 to 6. It is considered that this was because the binder covered the powder catalyst, and the exposed area of the powder catalyst decreased.

### Experimental Example 5: Sintering of Powder Catalyst Molded Article

<Sintering Conditions>

The powder catalyst molded articles of Examples 1 to 6 and Comparative Examples 2 to 6 were sintered. The sintering was performed under the following four conditions. 4 powder catalyst molded articles were produced in each of Examples 1 to 6 and Comparative Examples 2 to 6, and then sintered respectively under the following conditions:
Condition 1 . . . 30° C., 8 hours
Condition 2 . . . 80° C. 1 hour
Condition 3 . . . 120° C., 30 minutes
Condition 4 . . . 150° C., 10 minutes

<Immobilized State of Powder Catalyst after Sintering>

The powder catalyst molded article after sintering was fixed and was given a load using an air gun, and after that, the immobilized state of the powder catalyst was confirmed. In all of Examples 1 to 6, after the sintering under Conditions 1 to 4, the powder catalyst was immobilized without temperature dependence. In view of this, it was illustrated that even if the powder catalyst molded article was sintered, the immobilized state of the powder catalyst did not change, and no problem occurred by sintering. In addition, it can be said that since the sintering was performed at a temperature lower than the operating temperature (about 220° C.) of the Sabatier catalyst, the catalyst performance was also maintained.

### Experimental Example 6: Other Methods for Molding Powder Catalyst

### Comparative Example 7

TiO₂ particles serving as a carrier of the powder catalyst and water were mixed in a ratio of 1:10, and a slurry was obtained. The slurry was formed into pellets (size: 5 mm) and dried for 30 minutes at 80° C.

A water-IPA mixed solution (volume ratio of water 25%:IPA 75%) and a powder catalyst made from a TiO₂ catalyst supporting Ru formulated in the same method as Experimental Example 1 were mixed in a mixing ratio (weight ratio) of 10:1 to disperse the powder catalyst, and a slurry was obtained. This slurry was applied to the surface of the pellet and dried for 30 minutes at 80° C. Accordingly, powder catalyst pellets according to Comparative Example 7 were produced.

### Comparative Example 8

TiO₂ particles serving as a carrier of the powder catalyst and the powder catalyst made from the TiO₂ catalyst supporting Ru formulated in the same method as Experimental Example 1 were mixed in a ratio of 25:75, and a powder mixture was obtained. The water-IPA mixed solution (volume ratio of water 25%:IPA 75%) and the obtained powder mixture were mixed in a mixing ratio (weight ratio) of 10:1, and a slurry was obtained. The slurry was formed into pellets (size: 5 mm) and dried at 80° C. Accordingly, powder catalyst pellets according to Comparative Example 8 were produced.

### Comparative Example 9

The conditions of Comparative Example 9 were the same as those of Comparative Example 8 except that TiO₂ particles and a powder catalyst were mixed in a mixing ratio of 75:25, and powder catalyst pellets according to Comparative Example 9 were produced.

[Evaluation of Moldability]

When the powder catalyst pellets according to Comparative Example 7 were visually confirmed, the powder catalyst was desorbed from the pellet surface after drying, making it difficult to handle it. When the powder catalyst pellets according to Comparative Examples 8 and 9 were visually confirmed, the pellets lost their shape after drying. Accordingly, in the method that forms and dries the pellets as in Comparative Examples 7 to 9, the moldability of the powder catalyst was poor, and the molded article of such a powder catalyst may cause clogging when filled in the reactor.

### REFERENCE SIGNS LIST

10 Porous molding base material
20 Functional material molded article
100 Reactor
101 Reaction cell
102 Porous material

1. A method for producing a functional material molded article, comprising:
dispersing a functional material in a water-alcohol mixed solution to obtain a liquid dispersion;
impregnating a porous molding base material with the liquid dispersion to obtain an impregnated product; and
drying the impregnated product.

2. The method of claim 1, wherein the functional material is a hydrogen reduction catalyst for carbon dioxide.

3. The method of claim 2, wherein the hydrogen reduction catalyst for carbon dioxide has a structure in which catalytic metal nanoparticles and a metal oxide for suppressing grain growth of the catalytic metal nanoparticles are dispersed and supported on a carrier.

4. The method of claim 1, wherein a material of the porous molding base material is ceramic or metal.

5. The method of claim 1, wherein the porous molding base material has a porosity of 10% to 90%.

6. The method of claim 1, wherein the porous molding base material has a specific surface area of 0.5 to 10 m²/g.

7. The method of claim 1, wherein the ratio of the water-alcohol mixed solution to the functional material of the liquid dispersion is from 20:80 to 80:20 by weight.

8. The method of claim 1, wherein the ratio of water to alcohol in the water-alcohol mixed solution is from 5:95 to 95:5 by volume ratio.

9. The method of claim 1, wherein the porous molding base material has a plate shape, a disc shape, a rectangular parallelepiped shape, a cubic shape, a spherical shape, a hemispherical shape, a pyramidal shape, a conical shape, a cylindrical shape, or a combination thereof.

10. A functional material molded article in which a functional material is retained in pores of a porous molding base material, wherein the amount of the functional material retained is from 50 to 300 mg/cm³.

11. The functional material molded article of claim 10, wherein the functional material is a hydrogen reduction catalyst for carbon dioxide.

12. The functional material molded article of claim 11, wherein the hydrogen reduction catalyst for carbon dioxide has a structure in which catalytic metal nanoparticles and a metal oxide for suppressing grain growth of the catalytic metal nanoparticles are dispersed and supported on a carrier.

13. The functional material molded article of claim 10, wherein a material of the porous molding base material is ceramic or metal.

14. The functional material molded article of claim 10, wherein the functional material molded article has a plate shape, a disc shape, a rectangular parallelepiped shape, a cubic shape, a spherical shape, a hemispherical shape, a pyramidal shape, a conical shape, a cylindrical shape, or a combination thereof.

15. A reactor comprising a reaction cell filled with the functional material molded article according to claim 10.

16. The reactor according to claim 15, wherein an amount of the functional material retained in the reaction cell has a gradient.
